(19) United States
(12) Patent Application Publication
(10) Pub. No.: US 2008/0220062 A1
(43) Pub. Date: Sep. 11, 2008

(54) Sustained release of agents for localized pain management

(75) Inventor: Paul Ashton, Newton, MA (US)

Correspondence Address: ROPES & GRAY LLP, PATENT DOCKETING 39/41, ONE INTERNATIONAL PLACE, BOSTON, MA, 02110-2624, US

(73) Assignee: pSivida, Inc., Watertown, MA (US)

(21) Appl. No.: 11/977,148

(22) Filed: Oct. 23, 2007

Related U.S. Application Data
(60) Provisional application No. 60/853,658, filed on Oct. 23, 2006.

(51) Int. Cl. A61K 9/00; A61P 25/00

(52) U.S. Cl. 424/484

(57) ABSTRACT

The invention relates to the treatment of localized pain by providing sustained release of an agent suitable for treating pain, methods for preparing and administering the agent, and methods of formulating and administering the agent as a pharmaceutical preparation. The agent can be locally administered to reduce systemic concentrations of the agent.

### RELATED APPLICATIONS

This application claims the benefit of U.S. Patent Application No. 60/853,658, filed Oct. 23, 2006, which application is hereby incorporated by reference in its entirety.

### BACKGROUND OF THE INVENTION

Severe localized pain is associated with many physiological conditions, including, but not limited to, osteoarthritis, post-herpetic neuralgia, and post-surgical pain. Attempts to treat these conditions have included systemic administration of non-steroidal anti-inflammatory drugs (NSAIDS), steroids, and opioids. Unfortunately, such attempts often lead to systemic side effects while leaving insufficient drug present at the active site of the disease. As an example, a once a day oral sustained release formulation for morphine (Avinza®) is available for use in patients with osteoarthritis. Although effective at treating osteoarthritis, the formulation causes considerable side effects such as nausea, constipation, and vomiting. Such side effects often result from centrally mediated analgesia, produced through activation of opioid receptors in the central nervous system.

Intrathecal or epidural administration is marginally effective for reducing the side effects caused by some pain-relieving agents, but often only if reduced dosages are administered. Often, such reduced dosages are lower than is necessary or desirable to treat a patient's pain effectively. Accordingly, an improved system is needed for providing localized, sustained release of short-acting pain-relief drugs without heightening the side effects of such drugs. That is, an improved system is needed to deliver high, localized doses while maintaining low systemic plasma levels of the drug.

### SUMMARY OF THE INVENTION

The present invention provides for improved sustained release delivery of short-acting pain-relief agents. In certain aspects, the invention provides for improved sustained release delivery of opioids alone or in conjunction with other therapeutics. In certain aspects, the invention includes providing high, localized therapeutic levels of a pain-relief agent while producing low systemic levels of the agent. In one aspect, the present invention provides for sustained release formulations of the agent. The invention also relates to pharmaceutical compositions comprising one or more pharmaceutically acceptable carriers, diluents, adjuvants, or excipients in combination with the pain-relief agent. In certain aspects, the invention provides for methods of providing localized delivery of the agent and formulations thereof to a patient in need.

### DETAILED DESCRIPTION OF THE INVENTION

The present invention provides for the sustained release delivery of pain-relief agents, in particular the sustained release delivery of therapeutically effective localized doses of the agents while producing minimal systemic concentrations thereof. In preferred embodiments, the agents have short blood plasma half-lives (i.e., they rapidly clear from the body upon reaching the blood stream).

In certain embodiments, the sustained release of a therapeutic agent may be achieved by delivering the agent in codrug or prodrug form. In other embodiments, the sustained release of the therapeutic agent may be achieved by delivering the agent in a polymer matrix. In still other embodiments, the agent may be delivered by a polymer-coated drug delivery device. In certain embodiments, sustained release may be achieved by using low-solubility pain-relief agents.

In certain embodiments, the agent can be delivered in a therapeutically effective dose in a controlled manner to a localized site within a body in need of treatment. For example, but without limitation, U.S. application Ser. No. 10/134,033, filed Apr. 26, 2002, which claims priority to U.S. application No. 60/286,343, filed Apr. 26, 2001, 60/322,428, filed Sep. 17, 2001, and 60/372,761, filed Apr. 15, 2002; and PCT Application No. US02/13385, filed Apr. 26, 2002, disclose various embodiments of sustained release formulations using codrugs, prodrugs, and combinations thereof. Such formulations may be usefully employed with the systems and methods described herein, and the entire disclosures of such references are incorporated herein by reference.

According to the present invention, the at least one agent and formulations thereof are locally delivered. In certain embodiments, the agent may be applied as a dermal product wherein the sustained release of the agent is achieved as the agent passes through the skin. For example, but without limitation, such a composition may be used for treating post-herpetic neuralgia, or surgical, traumatic, or other wounds.

In certain embodiments, the agents may be delivered in a sustained release manner through an insertable sustained release system, e.g., as a long-lasting implant for post-surgical application. In certain embodiments, the sustained release system may entail intraarticular injection. In certain embodiments, the sustained release system of the present invention may be employed to treat osteoarthritis, or other types of severe localized pain.

In certain embodiments, sustained release of the agent may be achieved over a long period of time. For example, but without limitation, sustained release may extend over at least 1 day, preferably over at least 3 days, or even at least a week or a month. In certain embodiments, sustained release delivery occurs over more than a month, preferably at least 6 months or a year, or even over a period of several years.

The present invention contemplates that the pain-relief agent will remain sufficiently concentrated in a particular bodily area to achieve localized pain relief. In preferred embodiments, the systemic concentration of the agent remains very low. In preferred embodiments, the systemic plasma concentration of the agent remains less than about 1 ng/ml, preferably less than about 0.1 ng/ml, and more preferably less than about 0.01 ng/ml or even less than about 0.001 ng/ml. In certain embodiments, the systemic plasma concentration of the agent does not exceed the localized therapeutic level of the agent. In preferred embodiments, the agent's systemic plasma concentration is less than 75% of the localized therapeutic level, or even less than 50%, or less than 25% thereof.

In preferred embodiments, the agent has a low half-life when released into the bloodstream. In certain embodiments, the agent's half-life in the bloodstream is less than about 2 hours. More preferably, the half-life in the bloodstream is less than about 30 minutes, preferably less than about 20 minutes, even less than about 15 minutes in certain embodiments.

In certain embodiments, the invention allows the administration of suitably high dosages of pain-relief agents. In preferred embodiments, the agent is administered in a dose ranging from about 10 ug/kg body weight to about 1 mg/kg body weight, preferably from greater than 75 ug/kg to about 500 ug/kg.

In certain preferred embodiments, the systemic levels of the agent remain sufficiently low as to not trigger a centrally mediated analgesic response. In certain embodiments, the agent is administered with reduced (or even non-existent) side effects when compared to the agent delivered by standard commercial systems. Such side effects may include, for example, addiction, dysphoria, constipation, nausea, sedation, pruritus, respiratory depression, seizure, dry mouth, urinary retention, myoclonus, and the like.

In preferred embodiments, compositions comprising the agents are substantially pyrogen-free.

In certain embodiments, any or all of agents may be chiral. In some embodiments, the agents are substantially enantiomerically pure.

In certain embodiments according to the present invention, the agent (or at least one constituent part thereof) is an alkaloid analgesic, such as an opioid analgesic. In preferred embodiments, the agent is a short-acting opioid with a rapid blood-plasma half-life. For example, the agent may be remifentanil, or any other compound having a comparable or even lower blood-plasma elimination half-life, including without limitation any pharmaceutically acceptable salts, esters, codrugs, prodrugs, and protected forms thereof. In other embodiments, suitable compounds may include heroin, alfentanil, or any other compound having comparable or even lower blood-plasma elimination half-lives, including without limitation any pharmaceutically acceptable salts, esters, codrugs, prodrugs, and protected forms thereof. The invention may also be applied to other pain-relief agents. For example, suitable alkaloid analgesics include desmorphine, dezocine, dihydromorphine, dimepbeptanol, eptazocine, ethylmorphine, glafenine, hydromorphone, isoladol, ketobenidone, p-lactophetide, levorphanol, moptazinol, metazocin, metopon, morphine, nalbuphine, nalmefene, nalorphine, naloxone, norlevorphanol, normorphine, oxmorphone, pentazocine, phenperidine, phenylramidol, tramadol, viminol, fentanyl, oxycodone, codeine, ketamine, and sufentanil, and pharmaceutically acceptable salts, esters, codrugs, prodrugs, and protected forms thereof. Certain codrug embodiments may include, for example, morphine/morphine codrugs or morphine/diclofenac codrugs.

As mentioned, in certain embodiments, acceptable salts, esters, codrugs, prodrugs and protected forms of the foregoing may be used, as may any active metabolites of the foregoing (e.g., morphine-6-glucuronide), and specific enantiomers thereof (e.g., S-ketamine).

U.S. Pat. Nos. 5,919,473 and 5,589,480 disclose various embodiments for achieving sustained release delivery of opioids. Such disclosures may be usefully employed with the systems described herein, and the entire disclosures of those references are incorporated herein by reference.

In certain embodiments, the agent may be deployed, for example, in connection with an implant, a polymer implant, a gel, or even a rod or pellet consisting essentially of the agent. In certain embodiments, the agent may be deployed on a stent or other device. Such devices include, but are not limited to, surgical screws, prosthetic joints, artificial valves, plates, pacemakers, and the like. In certain embodiments, the agent may be deployed through, for example, dressings, bandages, adhesive strips, trocars, staples, sutures, surgical gauze, etc. In certain embodiments, the agent is formulated in a cream, in certain embodiments in a hydrogel.

In certain embodiments of the present invention, the compounds are delivered through a bioerodible drug delivery device capable of delivering one drug or even two or more synergistic drugs over a prolonged period.

In preferred embodiments, the device allows delivery of the compounds over a period of at least 3 hours, preferably at least 12 hours, or even 1 day, at least 2 days, or even at least 1 week, 1 month, or 1 year. In certain embodiments, the device is formed of a bioerodible polymer matrix selected from polyanhydride, polylactic acid, polyglycolic acid, polyorthoester, polyalkylcyanoacrylate, and derivatives and copolymers thereof. In other embodiments, the device may be non-bioerodible, for example comprising a non-bioerodible polymer matrix selected from polyurethane, polysilicone, poly(ethylene-co-vinyl acetate), polyvinyl alcohol, and derivatives and copolymers thereof. In preferred embodiments, the non-bioerodible device allows delivery of the compounds over a period of at least 1 day, preferably at least 2 days, or even at least 1 week, 1 month, or 1 year.

For example, but without limitation, U.S. Pat. Nos. 5,378,475, 5,773,019, 5,902,598, 6,001,386, and 6,375,972 disclose various embodiments of sustained release drug delivery devices. Other examples may include the devices taught in U.S. patent application Ser. Nos. 10/428214, 60/482677, 60/501947, and 60/483316, and PCT application US03/13733. These and other devices may be usefully employed with the systems described herein, and the entire disclosures of those references are incorporated herein by reference.

In another aspect, the invention contemplates administering the agents, compositions, and devices discussed herein to a patient. Certain aspects of the invention provide for localized, sustained release of the agents to facilitate this administration. For example, where codrugs are used, the agent contains two moieties linked together, e.g., through carbamate, carbonate, ester, or other bonds linking the molecules, which decreases the solubility of the codrug relative to one or both of the unlinked constituent compounds in aqueous solutions such as bodily fluids. In some of such embodiments, the codrugs have a high degree of chemical or enzymatic lability at physiological pH 7.4. A combination of low solubility and high chemical or enzymatic lability at physiologic pH provides that codrugs may be inserted at or near the locus of desired therapeutic activity, where they will be released slowly into the surrounding tissue and quickly converted into the active constituent compound upon exposure to physiologic conditions, thereby producing a high local concentration of the constituent compound. Because systemic administration is avoided by this method, the systemic concentrations of the residues may remain low, while the localized concentrations may be maintained within the therapeutic range over a period of time ranging from days to months.

The agents may be administered locally to a patient in need thereof. For example, the agents may be delivered in injectable form, such as in aqueous solutions, liposomes, emulsions, liquids, suspensions and microsphere nanoparticles. Preparation of such forms are known to those skilled in the art. See Remington's Pharmaceutical Sciences, 18th Ed., Mack Publishing Co., Easton, Pa., 1990, pp. 1504-1712, incorporated herein by reference. The agents may also be administered by rectal, parenteral (subcutaneous, intravenous, intramuscular), intrathecal, transdernal, and other such forms of administration. In certain embodiments, the agents may be applied orally in a topical manner (as opposed to by ingestion) for treatment of pain inside a patient's mouth. Suitable dosage forms may include dispersions, suspensions, solutions, capsules, elixirs, aerosols, patches, and the like. In some preferred embodiments according to the invention, one or more compressed pellets of an agent are inserted into the target tissue, for instance by subcutaneous or intramuscular injection.

Agents and compositions produced according to the invention can also be produced as therapeutics and can be delivered through a pharmaceutically acceptable carrier. Carriers such as starches, sugars, microcrystalline cellulose, diluents, granulating agents, lubricants, binders, disintegrating agents and the like may be used.

The person skilled in the art will recognize that additional embodiments according to the invention are contemplated as being within the scope of the foregoing generic disclosure, and no disclaimer is in any way intended by the foregoing, non-limiting examples.

All patents, publications, and references cited in the foregoing disclosure are expressly incorporated herein by reference.

### Terms and Definitions

As used herein, the term “insert” means insert, inject, implant, or administer in any other fashion. The term “inserted” means inserted, injected, implanted, or administered in any other fashion. The term “insertion” means insertion, injection, implantation, or administration in any other fashion. Similarly, the term “insertable” means insertable, injectable, implantable, or otherwise adminstrable.

The term “patient,” as used herein, refers to either a human or a non-human animal.

As used herein, the term “pain-relief agent” refers to any opioid, including any codrug, prodrug, or other pharmaceutical compound containing an opioid, or a combination of any of the foregoing. The term also refers to any formulation of any of the foregoing. The term also refers to a drug composition containing at least one of the foregoing together with at least one or more additional therapeutics. In certain embodiments, multiple therapeutically active agents may be delivered through the inventive formulations and methodologies.

As used herein, the term “EC₅₀” means the effective concentration of a drug, it being a dose of a drug that produces 50% of its maximum response or effect.

The phrase “pharmaceutically acceptable carrier” as used herein means a pharmaceutically acceptable material, composition or vehicle, such as a liquid or solid filter, diluent, adjuvant, excipient, solvent or encapsulating material, involved in carrying or transporting a subject drug to a particular location within the body. Each carrier must be “acceptable” in the sense of being compatible with other ingredients of the formulation and not injurious to the patient. Some examples of materials which can serve as pharmaceutically acceptable carriers include (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations. Preferred carriers are non-pyrogenic, i.e., do not substantially elevate the body temperature of a patient receiving the formulation.

The term “codrug” as used herein means a compound, or a prodrug form thereof, comprising a first molecule residue associated with a second molecule residue, wherein each residue, in its separate form (i.e., in the absence of the association), is an active agent or a prodrug of an active agent. The association between said residues can be either ionic or covalent and, in the case of covalent associations, either direct or indirect through a linker. The first molecule can be the same or different from the second. Codrugs, as that term is used herein, are more fully described in U.S. Pat. No. 6,051,576, the disclosure of which is incorporated herein in its entirety.

The term “localized delivery”, also referred to as “locally delivered,” refers to the delivery of a pain-relief agent to a location in the body experiencing pain. Such locations may include, without limitation, a bone joint, a particular span of muscle tissue, a span of skin, a site on a bone, or any other body site that can experience pain.

The term “prodrug” as used herein means a first residue associated with a second small molecule residue, wherein one of the residues is not biologically active. In preferred embodiments, one or both of the residues is a small molecule. In some embodiments, the prodrug may be biologically inactive in its prodrug form. The association between said residues is covalent and can be either direct or indirect through a linker. Prodrugs of biologically active compounds include esters, as well as anhydrides, amides, and carbamates that are hydrolyzed in biological fluids to produce the parent compounds.

The term “covalently linked” as used herein means either a direct covalent bond between two species, or an indirect association where two residues are not directly bonded but are both covalently bonded to an intermediate linker.

The term “substantially pyrogen-free” means a pharmaceutical composition having a pyrogen (e.g., endotoxin) concentration of less than about 0.3 EU/ml, preferably less than about 0.03 EU/ml, or even 0.01 EU/ml. The term also refers to a compound having a pyrogen contaminant (e.g., endotoxin) concentration of less than about 0.3 EU/mg, preferably less than about 0.03 EU/mg, or even 0.01 EU/mg.

The phrase “protecting group” or “protective group” as used herein means a temporary substituent that protects a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 2^{nd} ed.; Wiley: New York, 1991).

In general, “low solubility” means that the agent is only very slightly soluble in aqueous solutions having pH in the range of about 5 to about 8, and in particular to physiologic solutions, such as blood, blood plasma, etc. Some agents, e.g., low-solubility agents, will have solubilities of less than about 5 mg/ml, less than about 1 mg/ml, preferably less than about 100 μg/ml, or even less than about 20 μg/ml. More preferably, the solubility is less than about 15 μg/ml, and even more preferably less than about 10 μg/ml. Solubility is in water at a temperature of 25° C. as measured by the procedures set forth in the 1995 USP, unless otherwise stated. This includes compounds which are slightly soluble (about 10 mg/ml to about 1 mg/ml), very slightly soluble (about 1 mg/ml to about 0.1 mg/ml) and practically insoluble or insoluble compounds (less than about 0.1 mg/ml, preferably less than about 0.01 mg/ml).

What is claimed is:

1. A sustained-release formulation comprising at least one pain-relief agent having a blood plasma half-life of less than about 15 minutes under physiological conditions, said formulation being adapted for local delivery.

2. The formulation of claim 1, wherein a concentration of the at least one agent in a patient's blood plasma remains less than about 10 ng/ml when administered to said patient.

3. The formulation of claim 1, wherein a concentration of the at least one agent in a patient's blood plasma is less than a therapeutic concentration normally required for a systemic effect of the at least one agent.

4. The formulation of claim 1, wherein the at least one agent is in a dose of more than 75 ug/kg body weight of a patient to whom the at least one agent is administered.

5. The formulation of claim 1, wherein release of the at least one agent occurs over a period of at least 3 hours.

6. The formulation of claim 1, wherein the at least one agent is administered in a polymer matrix.

7. A sustained release formulation comprising at least one pain relief agent having a blood plasma half life of less than about 15 minutes, the agent having an aqueous solubility of less than about 5 mg/ml, said formulation being adapted for local delivery.

8. The formulation of any of claims 1-7, wherein the formulation is affixed to a drug delivery device.

9. The formulation of any of claims 1-7, wherein the at least one agent is a short-acting opioid or an active metabolite of an opioid.

10. The formulation of any of claims 1-7, wherein more than one pain-relief agent is delivered.

11. The formulation of any of claims 1-7, wherein the at least one agent does not trigger a centrally mediated analgesic response when applied to a body.

12. The formulation of any of claims 1-7, wherein the at least one agent gives rise to at least one reduced side effect when compared to the side effects produced by the at least one agent delivered by standard commercial systems.

13. A method for treating a patient experiencing pain, comprising:
providing a sustained-release formulation having at least one pain-relief agent in a polymer matrix, said at least one agent having a blood-plasma elimination half-life of less than about 15 minutes under physiological conditions; and
locally delivering said sustained-release formulation to the patient.

14. The method of claim 13, wherein the at least one agent's concentration remains less than about 10 ng/ml in the patient's blood plasma.

15. The method of claim 13, wherein sustained release of the at least one agent occurs over a period of at least 3 hours.

16. The method of claim 13, wherein a concentration of the at least one agent in a patient's blood plasma remains less than a therapeutic concentration normally required for a systemic effect of the at least one agent.

17. The method of claim 13, further comprising applying a dose of the at least one agent of more than 75 ug/kg body weight of the patient.

18. The method of claim 13, further comprising administering the at least one agent in a polymer matrix.

19. The method of any of claims 13-18, further comprising affixing the formulation to a drug delivery device.

20. The formulation of any of claims 13-18, wherein the at least one agent is a short-acting opioid or an active metabolite of a short-acting opioid.

21. The method of any of claims 13-18, wherein more than one pain-relief agent is delivered.

22. The method of any of claims 13-18, wherein the at least one agent does not trigger a centrally mediated analgesic response in the patient.

23. The method of any of claims 13-18, wherein the at least one agent gives rise to at least one reduced side effect when compared to the side effects produced by the at least one agent delivered by a standard commercial system.

24. A method for treating a patient experiencing pain, comprising:
providing a sustained-release formulation having at least one short-acting opioid in a polymer matrix, wherein the sustained-release of the opioid occurs over a period of at least three hours, and wherein the opioid has a systemic concentration sufficiently low to avoid triggering a centrally-mediated analgesic response in the patient, and locally delivering said sustained-release formulation to said patient.

25. The method of any of claims 13-18 or 24, wherein the at least one agent has a blood-plasma elimination half-life of less than about 10 minutes.
